(19) United States
(12) Patent Application Publication
(10) Pub. No.: US 2015/0250628 A1
(43) Pub. Date: Sep. 10, 2015

(54) IMPLANT

(71) Applicant: Phenox GmbH, Bochum (DE)

(72) Inventors: Hermann Monstadt, Bochum (DE); Ralf Hannes, Dortmund (DE); Dennis Herklotz, Wuppertal (DE)

(73) Assignee: Phenox GmbH, Bochum (DE)

(21) Appl. No.: 14/422,494

(22) PCT Filed: Aug. 22, 2013
(86) PCT No.: PCT/EP2013/067439

§ 371 Date: Feb. 19, 2015

(30) Foreign Application Priority Data
Aug. 22, 2012 (DE) 10 2012 016 555.2
Jan. 11, 2013 (DE) 10 2013 000 288.5
Apr. 16, 2013 (DE) 10 2013 006 503.8

(51) Int. Cl. A61F 2/89; A61F 2/90

(52) U.S. Cl. CPC A61F 2/89; A61F 2/90; A61F 2002/823

(57) ABSTRACT

The invention relates to an implant to be used for the occlusion of aneurysms in the region of vessel branches, in particular bifurcation aneurysms (A), with a mesh structure (3, 4), said implant comprising—from proximal to distal—a fixing section (b) by means of which the implant can be supported on the wall of a vessel, a permeable section (c) for the region of the vessel bifurcation, and a distal section (d) in which the implant is radially expanded in comparison to section (b) and which is intended for placement into the aneurysm (A). In the area of sections (c) or (d) a separation zone (T1, T2) is arranged that closes off at least partially the neck of the aneurysm, the distal section (d) being provided with a plurality of filaments and/or loops (12) connecting to section (c) and said filaments/loops (12) forming an angle of between −45° and +175° in relation to the longitudinal axis of the implant (1), wherein a positive angle is indicative of filaments pointing radially outward and a negative angle of filaments/loops (12) pointing radially inward. Alternatively, the distal section (d) may also be enlarged in the form similar to a sphere, mushroom, anchor or ellipsoid.

The invention relates to an implant to be used for the occlusion of aneurysms in vessel branches, in particular bifurcation aneurysms. Using a catheter and guidewire such an implant is to be transported to the placement site for the purpose of implanting it permanently. Accordingly, the invention also relates to such an implant which is attached to a guidewire so as to be ready for implantation. Furthermore, the invention relates to a method for placing the implant in position.

Arteriovenous malformation may significantly impair a patient and may even result in fatal risks. In particular, this applies to aneurysms, especially when these are found to exist in the cerebral region. Usually it is attempted to occlude malformations of this nature by means of implants. Such implants are as rule placed by endovascular methods using catheters.

Especially when treating cerebral aneurysms implanting platinum spirals has proven its worth, said spirals fill the aneurysm more or less completely, largely obstruct the blood inflow and enable a local thrombus or clot to form which fills 2o and ultimately closes off the aneurysm. Nevertheless, this treatment approach only suits aneurysms that have a relatively narrow access to the vessel system, so-called aciniform aneurysms. In the event of vessel protuberances having a wide access to the blood vessel there is a risk that the implanted spirals may be flushed out again and cause damage to other areas of the vascular system.

In such cases it has already been proposed to place into position a kind of stent that “bars” the opening of the aneurysm and in this way prevents the occlusion coils from being flushed out. Such stents are designed to have a relatively wide-mesh wall and have already been employed to treat some forms of aneurysms.

Vessel branches, in particular vessel bifurcations are a quite frequently occurring phenomenon. In case of a weak vessel wall the blood stream through an artery that acts on the front wall in a bifurcation quickly causes a protuberance or bulge which is prone to rapidly dilate further. More often than not, such bifurcation aneurysms have a wide neck which prevents a therapy to be performed with occlusion coils only.

Moreover, stent structures are missing that are conducive to “barring” the entry opening to the aneurysm in the region of vessel branching. With this in mind, it is the objective of the present invention to provide an implant capable of being used especially in the region of bifurcation aneurysms where it serves to “bar” the access opening of an aneurysm. By means of occlusion coils subsequently introduced the aneurysm can then be closed off.

“Barring” the aneurysm in this way is also conceivable with a view to influencing the flow of blood to reduce the number of occlusion coils or even bring it down to zero.

This objective is reached by providing an implant having a mesh structure comprising—from proximal to distal—sections (b) to (d):

A fixation section (b) by means of which the implant can be supported on a vessel wall,

a permeable section (c) for the vessel bifurcation area and
a distal section (d) where in comparison with section (b) the implant is expanded radially and which is meant to be placed into the aneurysm,
wherein in the area of sections (c) or (d) a separation zone is arranged that closes off at least partially the neck of the aneurysm, the distal section (d) being provided with a plurality of filaments and/or loops connecting to section (c) and said filaments/loops forming an angle of between −45° and +175° in relation to the longitudinal axis of the implant, wherein a positive angle is indicative of filaments pointing radially outward and a negative angle of filaments/loops pointing radially inward.

The terms “proximal” and “distal” are to be understood such that they refer to parts of the implant that point towards the guidewire and thus towards the catheter and attending physician (proximal), or as the case may be to parts that point away from the guidewire or attending physician (distal). Accordingly, proximal refers to items facing the guidewire whereas distal means facing away from the guidewire. The term “axial” refers to the longitudinal axis of the implant extending from proximal to distal while the term “radial” denotes levels/planes extending vertically thereto.

The implant according to the invention is provided with a mesh structure which may consist of a braiding of individual wires, with a mesh structure cut from a tube or with a mesh structure being a combination of the two. In that regard, the implant in general is to be viewed as a stent or stent-like object distinguished by its specialized way of application and design. In the event a braiding of individual wires is provided a number of between 4 and 24 wires is preferred for the sections (b) and (c).

The inventive implant is divided to form at least three but preferably four sections, i.e. the sections (a) to (d) as viewed from proximal to distal, with section (a) being optional. The sections (b) and (c) may be of identical design and differ only with respect to the position within the vessel when placement has been completed.

Section (a) is a tapering proximal section in which the mesh structure will be brought together in the form of one or several coupling elements. Said coupling elements are preferably situated at the periphery, i.e. when placement is done are arranged at the vessel wall when the implant has assumed its expanded form, and said elements serve to connect to an introducer sheath, in particular a guide or pusher wire. For application related reasons as well a centered arrangement is not considered expedient because a peripheral location of the coupling element(s) enables the implant to be retracted into the placement catheter more easily in the event of a misplacement. Embodiments provided with one or two coupling elements are preferred. Preferably, the coupling elements consist of coupling wires.

The coupling elements, especially the coupling wires, respectively the proximal end of the implant (without introducer sheath) may form an angle of between 0° and +60° in relation to the longitudinal axis of the implant, wherein a positive angle denotes a proximal end pointing outwards. Preferred is a range of between +10° and +30°, with the optimum angle depending on the configuration of the vessel. Such a positive angle facilitates an optimum expansion of the implant and enables the proximal end to be optimally located in the carrier vessel so that said proximal end is effectively prevented from projecting into the vessel lumen where it could interfere with the blood flow or insertion of another microcatheter. Preferably, the proximal end of the implant is of atraumatic design to make sure the vessel wall remains unharmed. Within the meaning of the invention it is to be understood that said angle configuration need not exist in non-implanted condition; important, however, is that the proximal end of the implant takes on such an angle after placement, i.e. it is sufficient to impress a desired deformation on the implant which it assumes after placement. In particular the use of shape memory materials is considered conducive in this context.

Section (b) serves for fixation and enables the implant to be supported on the wall of the vessel through which blood is led in. In this region, the vessel is undamaged and its wall capable of accommodating a stent wall. In the event of self-expanding implants, section (b) is automatically brought in contact with the vessel wall when the implant has been released whereas implants placed in position and dilated by means of balloons are pressed against the vessel wall in this area via a placement balloon.

Section (c) is a permeable section which may in particular have a greater mesh size than section (b) and is arranged and placed in the zone where the vessel bifurcation is actually situated. A greater mesh size allows a more or less uninhibited flow of blood through the meshes into the efferent vessel branches. However, it may not always be necessary to provide for a greater mesh width in section (c); if this is not the case sections (b) and (c) may also be largely or completely identical and differ only with respect to their position after placement in the vessel system.

In comparison to section (b) and usually also to section (c) the distal section (d) is radially enlarged outwardly. It is to be placed into the aneurysm itself and shall adapt to the widened out wall of the aneurysm.

In the area of sections (c) and (d), and in particular between sections (c) and (d) a separation zone is arranged which is to seal off the neck of the aneurysm. The separation zone shall in particular serve to retain occlusion means introduced into the bifurcation aneurysm. In case of a sufficiently impermeable separation zone sealing off the aneurysm neck to an adequate extent it might also be conceivable to dispense with any additional occlusion means such as coils. Of primary importance is that blood coagulation is ultimately achieved in the aneurysm. In any case the separation zone projects into the lumen of the implant orthogonally to the longitudinal axis. Coverage of the aneurysm neck ranges between 5 and 100%, with percentages between 30 and 60% being preferred. On the one hand, the surface coverage must be sufficiently great to either prevent any occlusion means introduced into the aneurysm from exiting the aneurysm or due to an adequate amount of material create an impermeable surface, but on the other hand a sufficient degree of flexibility of the implant must also be maintained to enable it to be introduced in the area of the bifurcation aneurysm.

However, also conceivable are other embodiments of the implant that are not provided with a separation zone but must nonetheless be regarded to fall within the scope of the invention. Such an implant may be used if more than one implant is to be introduced into the area of the bifurcation aneurysm, in particular two implants. This may prove advantageous in the event the aneurysm is of very irregular configuration and a closure is intended to be arranged only in a subzone of the aneurysm whereas the blood flow otherwise has to be maintained in another subarea due to the fact that aneurysm and discharging vessel overlap.

In this variant an implant without separation zone is first introduced, said implant otherwise corresponding completely with the one described herein. In a second step another implant provided with a separation zone is passed through the first implant to make sure the aneurysm can be closed off to the extent necessary. Through the placement of two implants complementing one another specific requirements that have to be observed in the treatment of an aneurysm can be met, if, for example, the distal sections (d) or permeable sections (c) of the implants are of different design.

The expansion or widening of section (d) is brought about by filaments and/or loops connecting to section (c). Said expansion/enlargement usually comprises at least two filaments/loops, in particular three or more filaments/loops. Typically, the number of filaments/loops ranges between 1 and 24, preferred are 2 to 6. Said filaments or loops may be made from appropriately formed wire elements but in the event the implant is cut from a tube may also be produced by adopting a laser cutting method to which said tube is then subjected. In case of loops these preferably consist of wire elements starting from section (c) then forming a loop and returning thereto, wherein said loops may basically be of optionally complex configuration. These may in particular be also three-dimensional objects depending on the shaping or arrangement of the loops. Loops are preferred because they are largely atraumatic and enable the sensitive vessel wall of the aneurysm to remain unharmed. However, other filaments may also be employed by means of which a radial expansion/enlargement of section (d) versus section (c) can be brought about. Said expansion may, for example, be of trumpet- or basket-like shape or provided in the form of a braiding. The filaments may be provided as braces that project radially outward, said braces are preferably concentrically aligned radially inwards. At the same time the braces may project in distal direction. For example, two or more braces may terminate in a common connection point at the distal end of section (c).

The angle the filaments/loops form in relation to the longitudinal axis of the implant after placement ranges between −45° and +175°, wherein a positive angle is indicative of filaments/loops pointing radially outward and a negative angle of filaments/loops pointing radially inward. In the event of relatively regular bifurcation aneurysms the angle preferably is in the range of between +45° and +90°; on the other hand, aneurysms are occasionally encountered that have an irregular shape, in particular are of highly asymmetric shape. In such cases it may prove expedient to make use of significantly deviating angles of the filaments/loops. It may be useful, for instance, to provide for a rather great angle in case the wall in one area of the aneurysm bulges considerably towards the blood supplying vessel. In such cases angles >90° are regarded expedient. In other cases it may be helpful to provide for part of the filaments/loops to point inwards, i.e. select negative angles to enable adaptation to the wall of the aneurysm. The angles the individual filaments/loops form may vary; in case of an asymmetric aneurysm it may, for example, be helpful to provide for some loops to be positioned at angles >90° whereas other loops form customary angles ranging between 45° and 90°. It is of importance that said angles are formed after placement has been completed; therefore, also an implant in which the angles indicated here have not yet formed when in a condition prior to implant placement, possibly due to external forces, is to be considered to fall within the scope of the invention.

Angles that the filaments/loops form in relation to the longitudinal axis of the implant may, for example, range between 45° and 90°, −45° and 0°, 90° and 135° or 135° and 175°.

The filament/loops in section (d) may be continuations of the wires or strings forming the remaining implant structure, but may as well be separate wire filaments attached in the distal region of the remaining implant structure, i.e. at the distal end of section (c), for instance through a laser welding technique. In this context, each filament and each loop of section (d) may be connected to the remaining implant structure via one or a plurality of connection points, in particular only one or two connecting points per loop/wire filament may be provided.

As per an alternative embodiment an implant having a mesh structure is proposed to be used for the occlusion of aneurysms in the area of vessel branches, particularly bifurcation aneurysms, which—from proximal to distal—is provided with sections (b) to (d), that is

a fixation section (b) by means of which the implant can be supported on a vessel wall,
a permeable section (c) for the vessel bifurcation area and
a distal section (d) in which the implant in comparison to section (b) is radially enlarged and which is destined for placement into the aneurysm, wherein in the area of sections (c) or (d) a separation zone is arranged that at least partially closes off the neck of the aneurysm, with said distal section (d) taking on an enlarged shape similar to a sphere, mushroom, anchor or ellipsoid. Preferably, the distal section (d) is not centrally but peripherally attached to section (c). With the exception of the deviating shaping of distal section (d), what has been said with reference to the embodiment according to claim 1 shall apply to all other aspects of the implant.

The forms mentioned hereinbefore are to be viewed as alternatives which may also be used to produce radially expanded section (d). A spherical section (d), for example, can well adjust itself to the inner wall of the aneurysm because a regular bifurcation aneurysm often exists basically in the form of a sphere. It is to be noted in this respect that within the scope of the invention a spherical form need not only be a true sphere as per its geometrical definition but may also be of deviating round three-dimensional shape which are deemed to be spheres as proposed by the invention. In some cases the form of section (d) is also comparable to an ellipsoid but it shall also be understood here that this need not be an exact spheroid in order to be regarded as ellipsoidal within the meaning of the invention. Moreover, sections (d) may also have mushroom- or anchor-like shapes which are in particular suitable for the treatment of irregular aneurysms, for example if a wall portion of an aneurysm shows significant bulging in the direction of the supplying vessel. In the event of a mushroom or anchor form this is achieved in that some areas of section (d) extend in proximal direction. It shall be understood here as well that a section of mushroom- or anchor-like shape may also be asymmetric, for example may have areas that only on one side extend in proximal direction. Provided the surface density of section (d) is sufficient, this section itself may be used as separation zone so that extra devices may be dispensed with where appropriate. Distal section (d) may be made by laser cutting techniques or of braided design, with between 8 and 128 wires being preferably employed.

The implants according to the invention may be manufactured from customary stent materials, for example consist of medical steel or cobalt-chromium alloys, however they consist in particular of shape-memory materials such as nitinol or ternary nickel-titanium alloys.

As mentioned hereinbefore, an implant according to the invention is preferably cut at least partially from a tube, in particular from a tube made of a shape-memory alloy. The separation zone as well can be cut out of the tube.

The separation zone provided in the inventive implant extends in particular between sections (c) and (d). It is to be noted in this context that section (c) at least within its distal end region may have an expanded shape as compared to section (b) which may be helpful in the event the bifurcation aneurysm has already formed in parts of the discharging blood vessels. In that case, the access portion of the aneurysm must be kept clear for the blood stream that branches off so that the separation zone extends within the aneurysm itself. The already enlarged portion of section (c) then merges into section (d), where it may expand further as the case may be. In this case as well the separation zone is located between sections (c) and (d). In case of a very shallow configuration of section (d) the separation zone may even coincide with section (d).

On the one hand, the separation zone may be designed to comprise introduced fibers, threads, thin wires, a membrane or similar separation elements but, on the other hand, may also be an integral part of the implant in the sense that the separation elements may be cut out of the basic tube and appropriately transformed or be composed of a wire braiding, for example in the shape of loops or strings. In the event of loops or strings these elements point radially inwards into the lumen of the implant, other than the loops of the distal section (d) that at least for the most part point outwards. To make sure the inwardly arranged loops/strings do not interfere with each other it may be expedient to have them designed asymmetrically. The number may vary depending on the structure of the implant and the number of honeycombs.

The threads making up the separation zone may be made of a polymer material, for example a polyamide such as nylon (polyhexamethylene adipic acid amide). Also possible is to use metal for this purpose with shape memory alloys being preferred, in particular nickel titanium alloys such as nitinol.

Another possibility is to provide a membrane in the separation zone, said membrane being largely or completely impermeable to blood and in this way capable of separating the aneurysm from the blood flow. In the event the aneurysm can very nearly completely be isolated from the blood flow an introduction of occlusion means into the aneurysm may, circumstances permitting, be dispensed with so that the separation zone in this case does not serve to retain occlusion means. The membrane may be attached to a braid of threads or wires, for example the threads or wires may form a structure over or onto which the membrane is spanned. Additionally, further threads/wires are conceivable which, for example, may extend or be arranged to form a cross or crosshairs. Nevertheless, an arrangement of threads or wires is not necessarily needed for this purpose, the separation zone may also be spanned without using additional threads or wires.

However, even in cases where a membrane is provided in the separation zone it may still be of advantage to additionally introduce occlusion means into the aneurysm. For this reason it may be expedient to provide the separation zone with a membrane that has one or several cutouts so that occlusion means, in particular coils, can be placed into the aneurysm through these cutouts. Said cutout shall be appropriately sized such that a catheter can be pushed through it into the area of the aneurysm, with the placement of the respective occlusion means being effected through the catheter. On the other hand, the cutout shall cover the neck of the aneurysm to such an extent that the occlusion means are prevented from exiting the aneurysm in an uncontrolled manner, with any threads/wires spanning the separation zone in this case may perform an additional retaining function. It goes without saying in such a case that the threads or wires must not be spaced too closely so as not to interfere with the positioning of a catheter and the introduction of the occlusion means.

To enable occlusion means to be introduced into the aneurysm the membrane spanning over the separation zone may also be designed so as to be pierceable partially, with such piercing being typically brought about by a microcatheter or guidewire. Through the opening so created a microcatheter is then run through which the occlusion means are placed in position. The membrane should be designed such that after it has been pierced it remains partially intact to ensure the occlusion means continue to be prevented by the membrane from exiting again. For example, threads or wires arranged in the separation zone in the form of crosshairs spanning said zone may ensure that only a segment of the membrane forms an opening when being pierced whereas the other segments of the membrane remain covered due to the fact that the marginal areas of the membrane are stabilized and safeguarded by the threads/wires against rupturing. The membrane spanning the separation zone may either be a single membrane which is pierced only partially or may consist of several smaller membranes.

Instead of or in addition to providing a membrane in the area of the separation zone it may turn out to be expedient to arrange membranes in the interior of the (wire)loops forming section (d). In the event membranes are placed in the separation zone and also inside the loops this will facilitate fixation of the membrane.

The membrane need not be limited to the separation zone and the interior of the loops but may span the totality of separation zone and loops as a result of which the loops may serve to hold the membrane in place. Membranes may be arranged, for example, in the spaces between the loops.

Even if section (d) is formed, wholly or in part, by filaments other than loops it is possible to arrange membranes in this location. For example, one or several membranes may be put up by means of braces projecting radially outwards. In such a case the structure resembles an umbrella, i.e. when section (d) expands the unfolding braces put up between them one continuous or several membranes. By providing a plurality of braces and in this way a corresponding number of brace ends a larger and more circular area can be covered by the membrane resulting in the interspaces to be reduced in size.

For the purpose of delimiting and reinforcing the membrane threads may also be spanned between the individual loops/filaments, that is, the membranes are limited at least partially at the sides by one or several threads serving to connect the loops/filaments with each other. Such a delimiting of the relevant membrane must not necessarily take place via a thread in every direction, even the loops/filaments themselves may to some extent serve this purpose. For example, the outer edge of the membrane which is often situated further distally may be bordered by threads while the inner edge be formed by loops/filaments. In comparison to a membrane without delimitation at the sides an additional protection of the membrane is achieved in this way so that damage and cracks can be avoided. The threads are preferably made of a polyamide such as nylon.

The provision of a membrane in the area of the separation zone is to be considered advantageous in that said membrane compactly folds together in distal or proximal direction in the catheter when the implant is placed so that an implant can be made available that in expanded condition has a largely impermeable separation zone and when in contracted state is capable of easily passing through narrow blood vessels as well. Otherwise, in comparison to an implant without separation zone the structure of the implant described hereinbefore is largely the same.

The membrane can be manufactured of a polymer material such as polytetrafluoroethylene, polyester, polyamides, polyurethanes or polyolefins. Especially preferred are polycarbonate urethanes. It is especially desirable to provide for an integral connection of the membrane with the threads or wires forming the separation zone. Such a connection may be achieved by coating the threads/wires by immersion or spraying techniques.

Preferably, the membrane is produced by an electrospinning process. By applying an electric current fibrils or fibers are separated from a polymer solution and deposited on a substrate. Said deposition causes the fibrils to agglutinate into a non-woven fabric. As a rule, the fibrils have a diameter ranging between 100 and 3000 nm. Membranes created by electrospinning have a very uniform texture and may embrace or include within a basic structure comprising threads or wires. The membrane is tenacious, withstands mechanical stresses, and can be pierced mechanically without an opening so created giving rise to cracks propagating from it. The thickness of the fibrils as well as the degree of porosity can be controlled by selecting process parameters as appropriate. In the context of producing the membrane and with respect to materials suitable for this purpose special attention is drawn to publications WO 2008/049386, DE 28 06 030 A1 and literature referred to therein.

Also of advantage is an implant that has a separation zone formed by a membrane which is in contact with the inner side of the implant, wherein said membrane in turn is permanently attached to further outer membrane segments filling out the individual loops. Such a membrane structure can be produced by electrospinning. The inner and outer membrane layers in this case are connected in part; in places where the inner membrane layer is not attached to the outer membrane layer it undergoes contraction similar to a nylon stocking and in this way forms a separation zone that can be passed through.

Instead of by electrospinning the membrane may also be produced by an immersion process.

The membrane must not necessarily be arranged orthogonally to the longitudinal axis of the implant but may also be oriented towards proximal. Although the membrane in its peripheral area is secured in this case to the circumference of the implant the middle region of the membrane, however, extends in proximal direction. In this way, a conical or pyramid shape is formed wherein the base of the cone/pyramid is oriented orthogonally to the longitudinal axis with the membrane in its peripheral region being attached to the implant whereas the apex of the cone/pyramid is situated further to proximal. In this manner, the flow of blood is divided and directed sideways when coming into contact with the membrane so that the ingress of blood into the aneurysm is largely prevented.

Even if the membrane forming the separation zone has a conical or pyramid shape, said membrane may also be provided with one or a plurality of cutouts to make sure occlusion means may continue to be introduced into the aneurysm through said cutouts after the implant has been placed in position.

To make sure the conical or pyramid shape of the membrane can be maintained on a permanent basis the membrane should be secured to a framework structure of threads or wires, but basically this structure may also consist of strings/lands cut, for instance by means of a laser, out of the structure forming the implant. Care must be taken in this case that the threads/wires are of adequate stiffness to prevent the membrane from undergoing reorientation or turning inwards as a result of the blood pressure. It may be necessary in this respect to introduce additional threads or wires.

Another possibility is to create crosshairs consisting of two relatively long individual threads to which the membrane is attached, with the membrane initially not being tensioned due to the length of the individual threads. Moreover, one or several threads may be attached to a further proximally situated loop of the implant so that the crosshairs and thus the membrane is spanned/tensioned in proximal direction as soon as the implant undergoes stretching. It shall be understood, however, that the crosshairs must not necessarily be composed of two threads only but other thread braidings of nearly unlimited configuration are conceivable as well that establish a type of framework impressing a structure onto the membrane.

Generally speaking, it is of importance for the invention that the separation zone performs its intended function which is to reliably retain occlusion means, for example occlusion coils, introduced into the aneurysm or deflect the flow of blood in such a manner that further occlusion means are not needed. The separation zone extends orthogonally to the longitudinal axis of the implant, with the fibers, threads, wires etc. forming said separation zone being essentially arranged in one plane.

If the separation zone is designed by introducing fibers, threads or thin wires it is considered expedient to arrange eyelets in the separation zone area. For example, the meshes of section (d) may be provided with relevant eyelets into which the threads are knotted in a crosswise or starlike fashion. The eyelets proper may be made of fiber material. The threads/fibers consist, for example, of a suitable polymer such as a polyamide (nylon) or be composed of metallic fibers.

However, the separation zone may also be created by means of curved elements cut from a tube material or of (wire)loops wherein the meshes of section (d) are deformed outwardly and the curved elements/loops of the separation zone bent inwardly into the body of the implant. At least one curved element/one loop is required. If between two and four curved elements/loops are used these will form a stable separation element which reliably retains the occlusion means introduced into an aneurysm.

The loops may be of honeycomb shape. When contracting the implant the loops are typically stretching in proximal direction and thus lean against the other filaments of the implant so that the implant may be easily moved through a catheter without causing problems. The separation zone which is formed by the loops may leave slot-like openings between the loops through which the occlusion means can be introduced into the aneurysm. Alternatively, it is also possible, however, to provide the loops and/or the interspaces between the loops with a membrane to enable an impermeable as possible separation zone to be achieved. Basically, membranes may also be used that are provided with one or several openings.

The distal section (d) of the implant provided by the invention is designed so as to be particularly atraumatic, soft, and elastic. Walls of aneurysms are rather delicate and may rupture when forces are applied so this must by all means be prevented. To this end, especially the distal section (d) of the inventive implant has to be designed so as to be atraumatic. This is achieved by an arrangement of loops, for example, that adjust gently to the wall of the aneurysm in places where they are in contact. Same as other regions of the implant such loops may be produced by laser cutting from a tube, created by means of tacked-on wires connected, for example, to section (c) by a laser welding method or produced by a uniform wire braiding. This zone of transition coincides in particular with the separation zone but may as well constitute an extended area of section (c) with the separation zone being arranged distally of it.

In any case, it is of great importance in the distal section (d) that all wire ends are formed in an atraumatic fashion to make sure aneurysm wall perforations cannot occur.

The meshes in the distal section (d) may terminate in rounded bends or arches, but especially at the distal end may also be provided in the form of protruding nose-shaped rounded off and in this way atraumatically designed spouts. These rounded spouts enable the implant located in elongated shape inside the catheter to be easier moved with less force having to be exerted.

The inventive implants may be provided in the form of a continuous laterally closed tube having a mesh structure but may also be slotted at the side either partially or all the way through. This slotted configuration may extend axially parallel or be of oblique/helical arrangement. In such a case, the mesh structure in the slotted areas is coiled up to suit the shape of the vessel, for example in the form of a rolled segment of a wire mesh fence. During placement, such a slotted implant is capable of suitably adapting to the vessel lumen, especially of the supplying vessel, with a slight underlap (gap) or overlap of the lateral edges of the mesh structure being as a rule viewed to be unproblematic.

A partial slot terminating at the distal section (d) may be provided, for example. Such a slotted arrangement permits good adjustment to the vessel configuration, in particular in the area of sections (a) to (c), and thus enables the implant to be well secured within the vessel. Surprisingly, it has been found that a slotted arrangement should not exert a negative influence on the radial force.

It is possible to provide at least some of the meshes of the implant with breaks, i.e. part of the meshes are not completely closed. Such an open-cell design affords higher flexibility which may offer benefits when treating highly tortuous blood vessels. Moreover, the omission of strings/braces will enhance the flow of blood in the area of the vessel branch. However, such an advantageously increased flexibility has a drawback in that it will be more difficult to retract an implant of open-cell design into the microcatheter in the event this becomes necessary during placement. For that reason, the proximal attachment to an introducer sheath via section (a) may be omitted with such an embodiment. An alternative introducer system may, for example, be designed such that the implant radially compressed within the microcatheter rests on a wire between two cams and automatically unfolds when the microcatheter is removed and in this manner disconnects from the introducer system.

As a rule, the implants according to the invention are provided with marker elements facilitating visualization and their positioning at the placement site. Marker element of this type are, for example, arranged in the area of the distal end of section (d), and may shape the connection points of joined wires so as to be non-traumatic. Such marker elements may also be provided in the form of wire windings, as sleeves and as slotted tube segments to be crimped onto the implant, for example in the transition region of sections (c) and (d) or to the wire loops of section (d). For said marker elements in particular platinum and platinum alloy materials are suitable, for example alloys of platinum and iridium, as they are frequently used in prior art for marking purposes and as material for occlusion coils. Ideally, the distal section (d) and in particular the loops/filaments are completely or in part radiopaque, i.e. they are made to be visible during radiography.

It is also possible to make use of radiopaque substances in the membranes. These may radiopaque particles as they are customarily employed as contrast medium for radiotechnological purposes. Such radiopaque substances are, for example, heavy metal salts such as barium sulfate or iodine compounds. A radiopaque membrane proves beneficial during implant placement and for localization purposes and may be used either additionally to or instead of marker elements.

If thought expedient, part of the honeycombs of the implant may be formed using braces of thinner cross section to increase the implant's flexibility. Preferable, the area is situated in section (b) and intended to meet requirements associated with an irregular blood vessel configuration in the fixation zone.

The implants must not necessarily be of tubular structure but may also be provided in the form of rolled up “mats” that are braced in position against the wall of the vessel. The implants may also be partially slotted.

Moreover, the invention relates to an implant in accordance with the description hereinbefore, said implant being coupled to a customary guidewire. Such an attachment may, for example, be brought about by means of connection elements dissolving electrolytically under the influence of electric current. Such connection elements and materials have often been described in particular for the severance of occlusion coils and stents. Also a mechanical detachment through coupling elements may be realized without difficulty, with such coupling elements appropriately interacting with suitably designed coupling parts of the guidewire. Under the external restraint of a catheter or enclosure this connection remains intact; however, after the implant and its coupling location have been released from the catheter or enclosure the attachment disconnects causing the implant together with the coupling elements forming part of the implant to be liberated.

The invention also relates to the placement of the inventive implants in the blood vessel system. This can be brought about with the help of a customary catheter or microcatheter which is a proven and frequently adopted technique. In case the aneurysm is not sufficiently sealed off already by the separation zone alone, occlusion means are introduced into the aneurysm after the implant has been placed in position. For this purpose, the distal end of a microcatheter is moved through the separation zone into the aneurysm following which the occlusion means, in particular coils, are released. When this has been done the microcatheter is retracted while the implant prevents the occlusion means from escaping from the aneurysm. Aside from customary occlusion means such as coils bodies of other shape and configuration may also be employed to the close off aneurysms, for example spherical bodies of a braided design or formed differently.

The invention is explained in more detail by way of the enclosed figures where

FIG. 1 is a schematic representation of a bifurcation aneurysm;

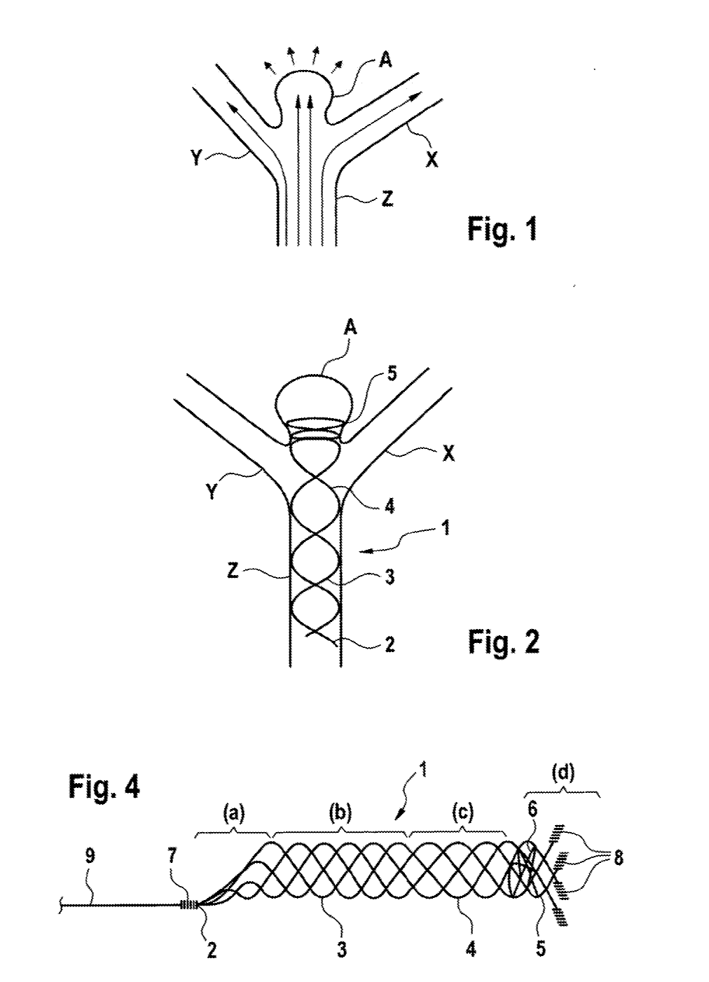

FIG. 2 shows schematically an inventive implant placed in the area of a vessel branch with bifurcation aneurysm;

FIG. 3a shows the basic principle of the inventive implant with its sections;

FIG. 3b is another schematic diagram of the inventive implant;

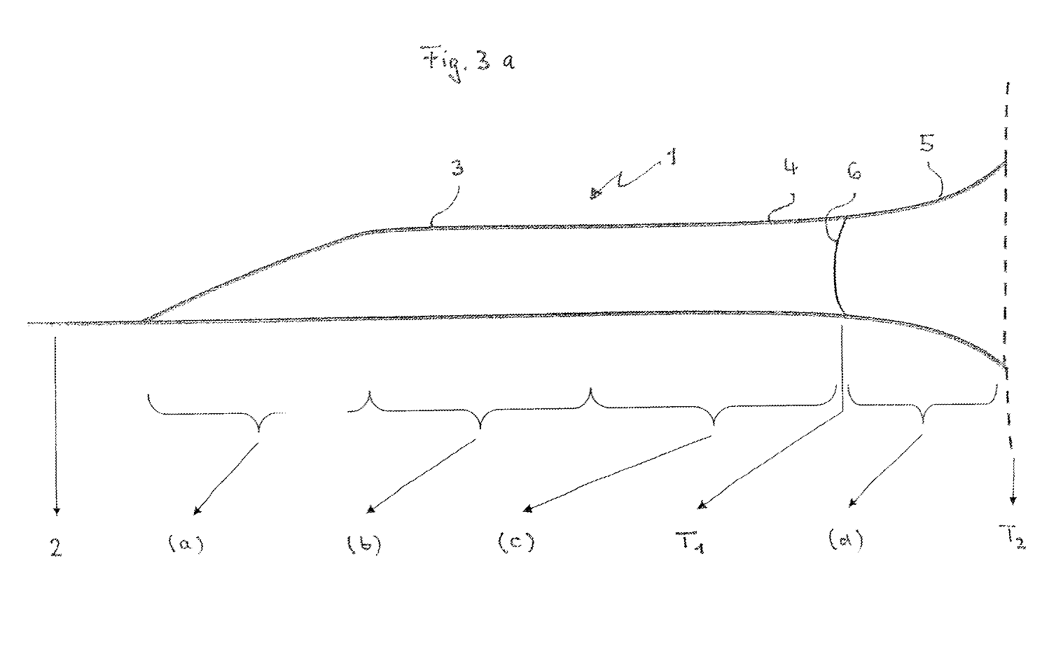

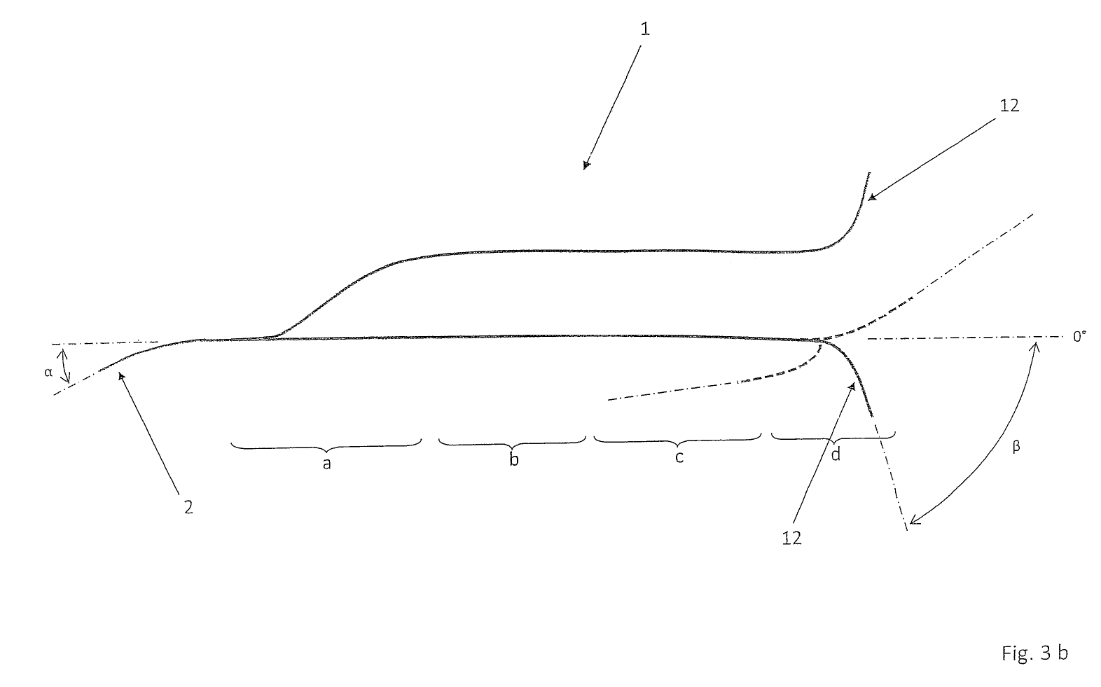

FIG. 4 illustrates an inventive implant as it can be employed as per FIG. 2;

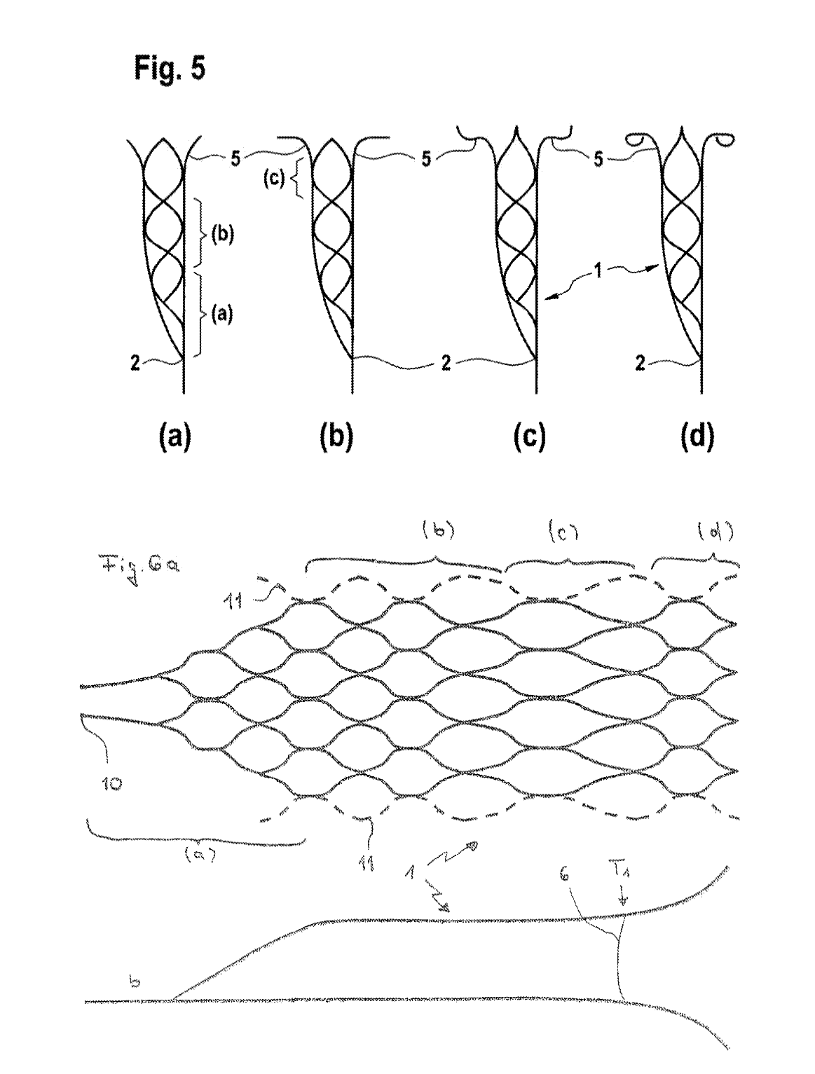

FIG. 5 shows variants of section (d) of an implant according to the invention;

FIG. 6 depicts a preferred embodiment of an inventive implant as a spread planar representation;

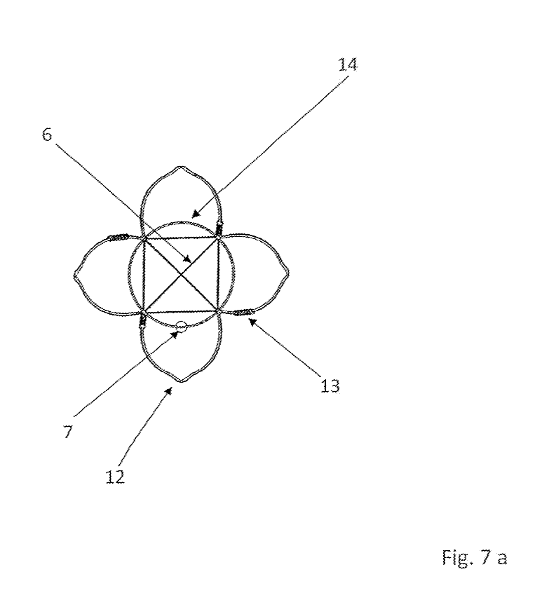

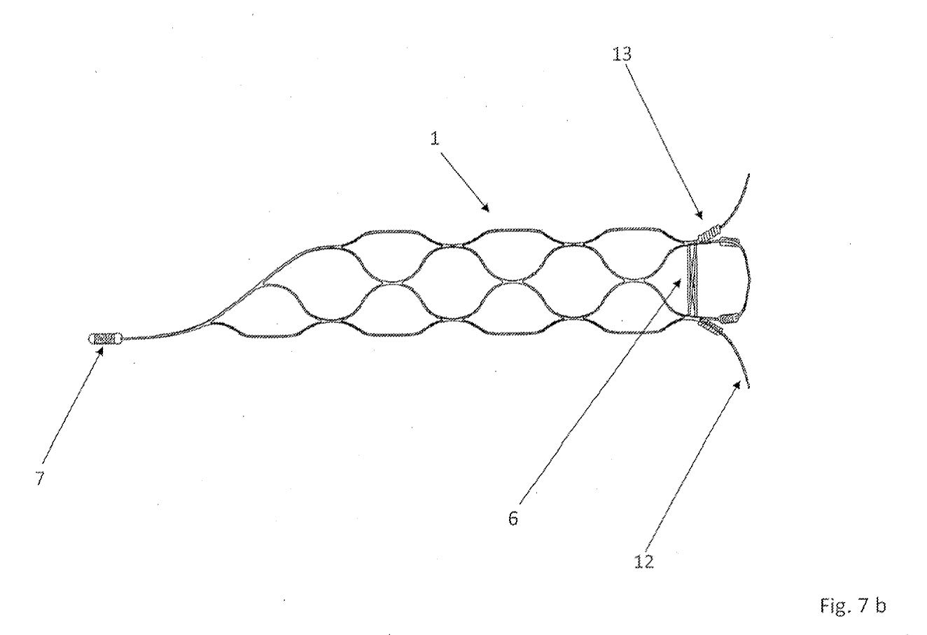

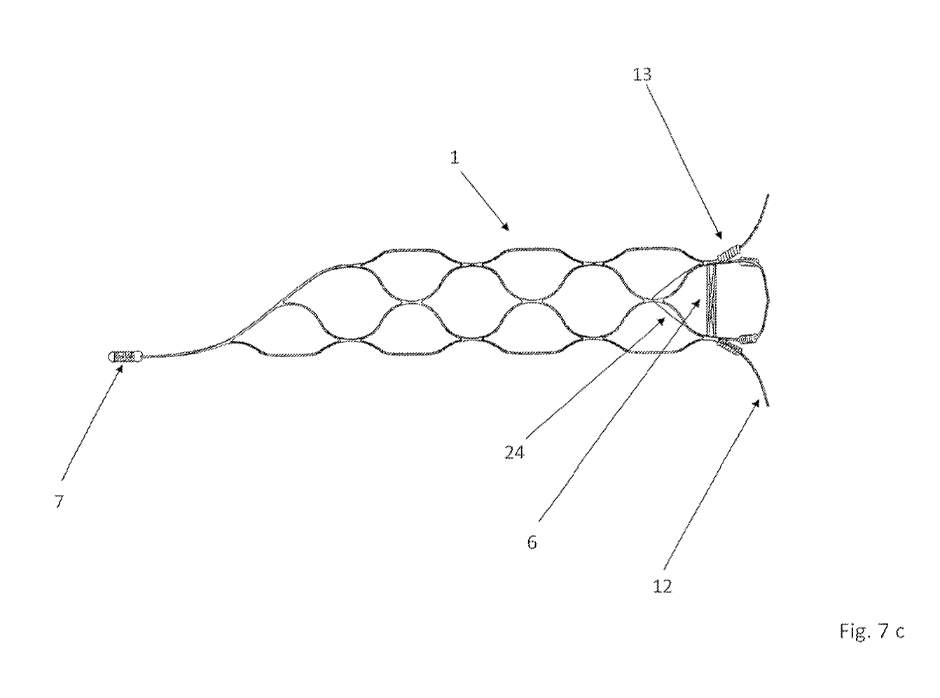

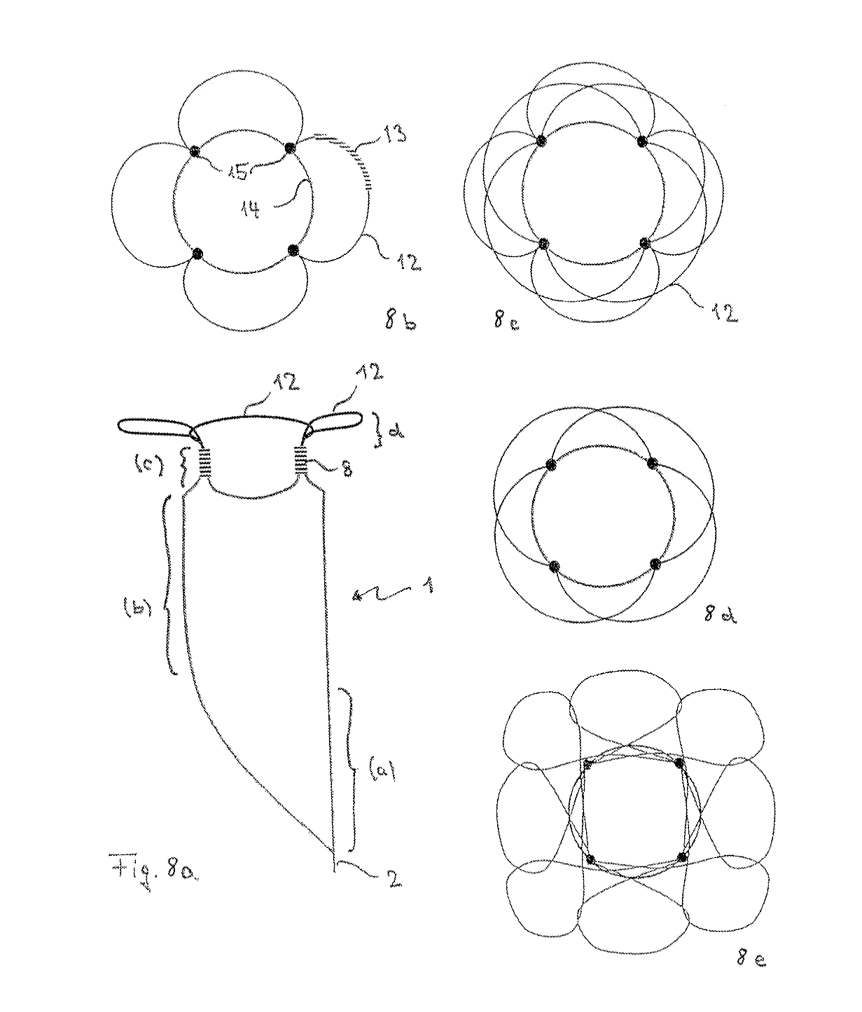

FIG. 7a shows an inventive implant with loop-like distal sections (d) viewed from distal direction;

FIG. 7b is a side view of the inventive implant shown in FIG. 7a;

FIG. 7c illustrates an inventive implant provided with a membrane in the separation zone extending in proximal direction;

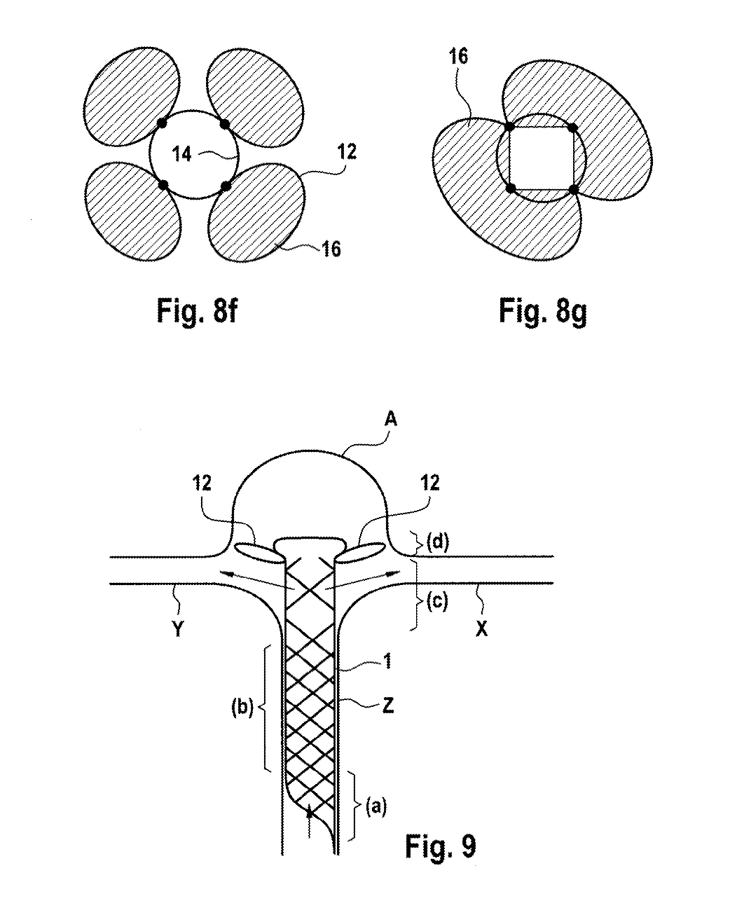

FIG. 8 shows additional variants of an implant according to the invention with loop-shaped distal sections (d);

FIG. 9 is the illustration of a bifurcation aneurysm with lateral vessels branching off the aneurysm area with an inventive implant in place;

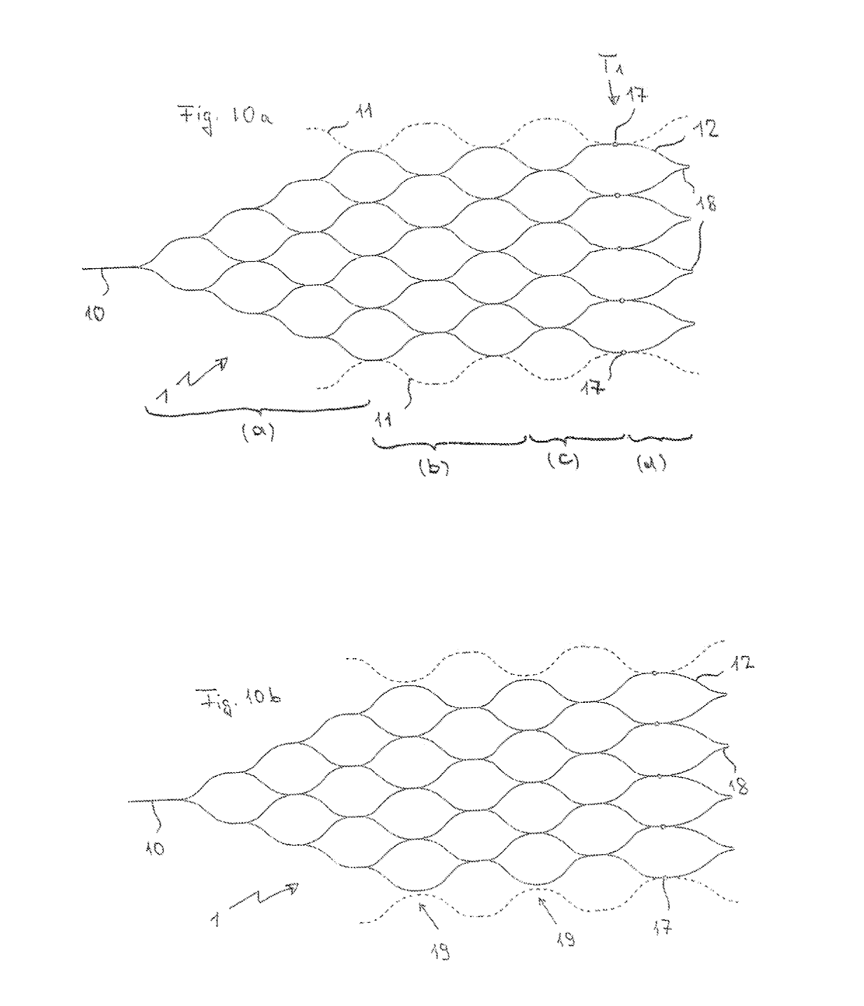

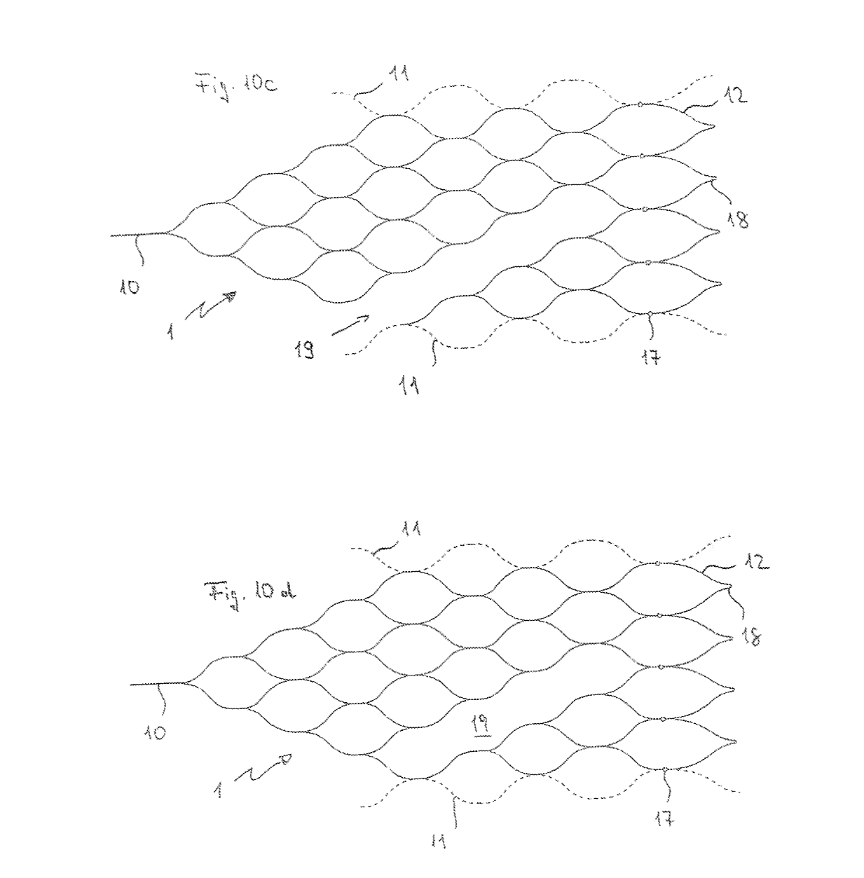

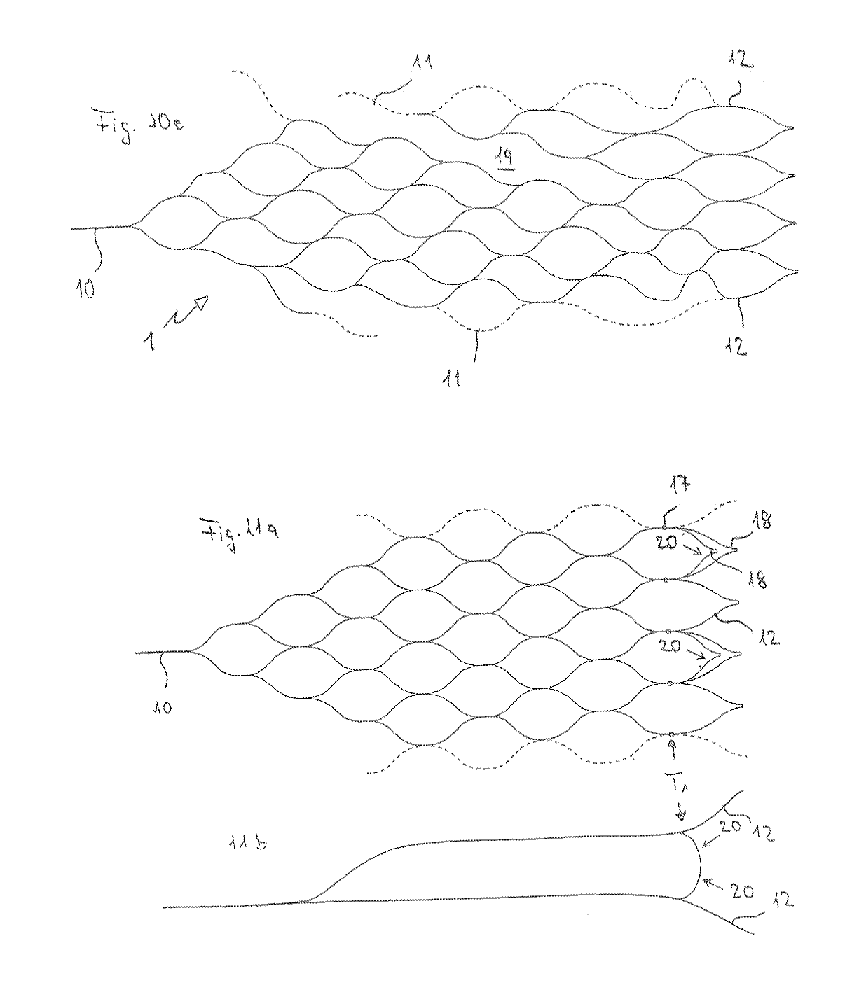

FIG. 10 is a spread planar representation of variants of implants according to the invention;

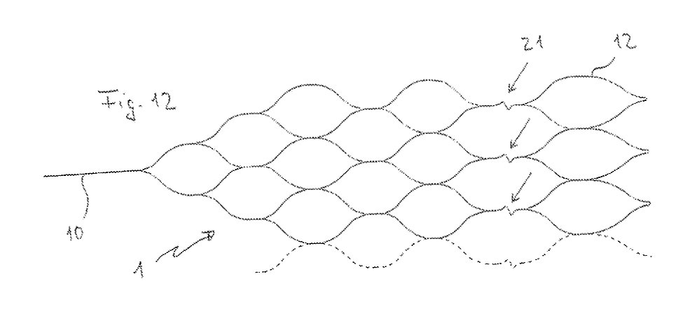

FIG. 11 shows another variant with inwardly and outwardly oriented curved elements in section (d);

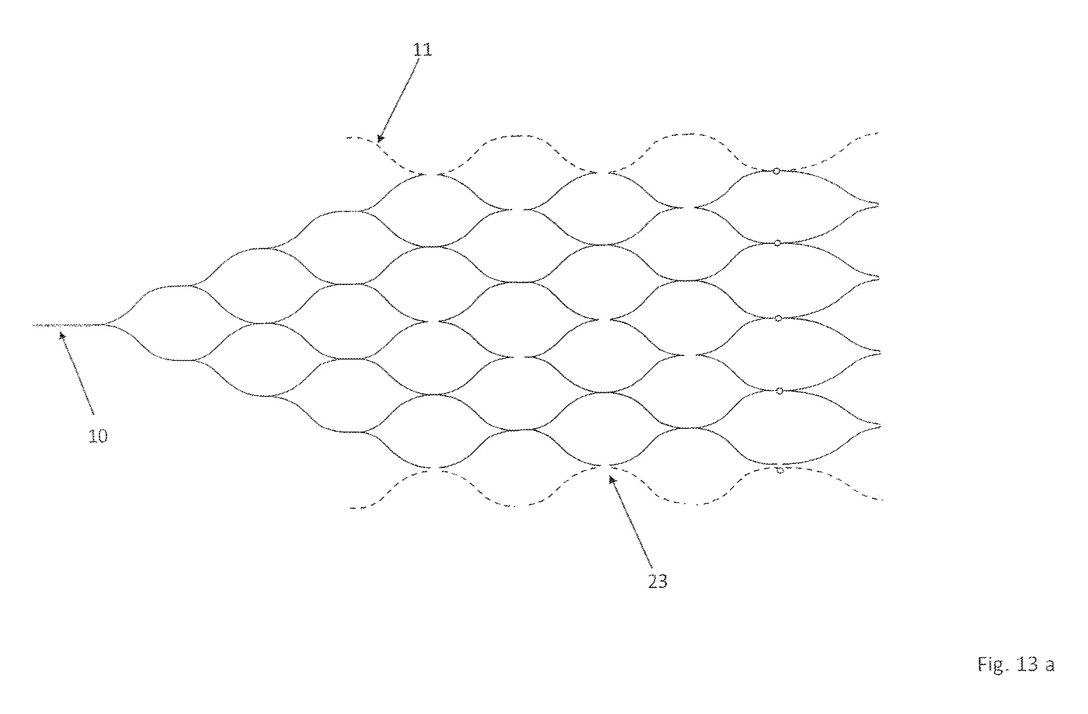

FIG. 12 shows another variant with articulated connectors in section (c);

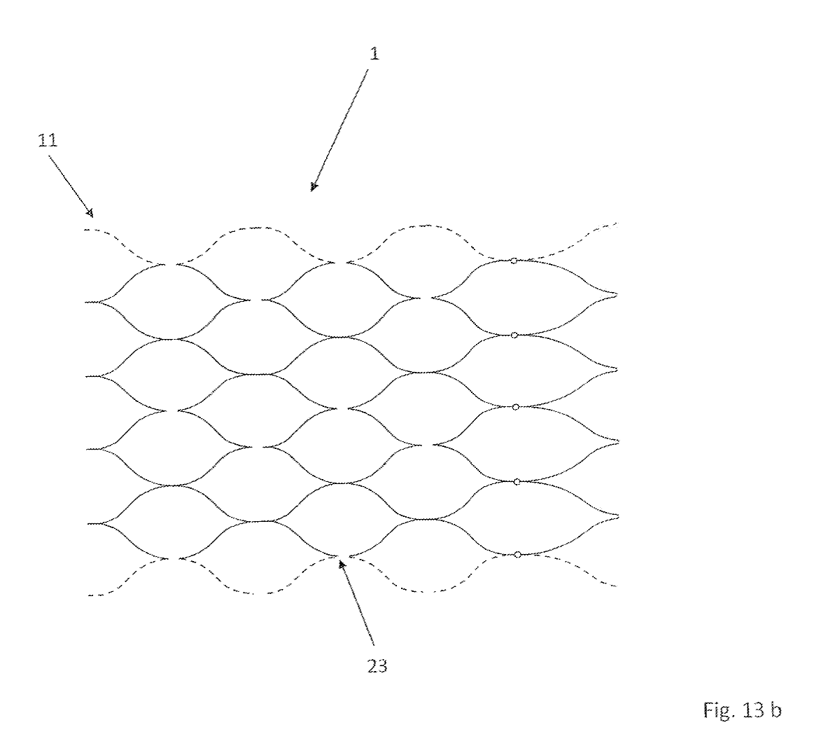

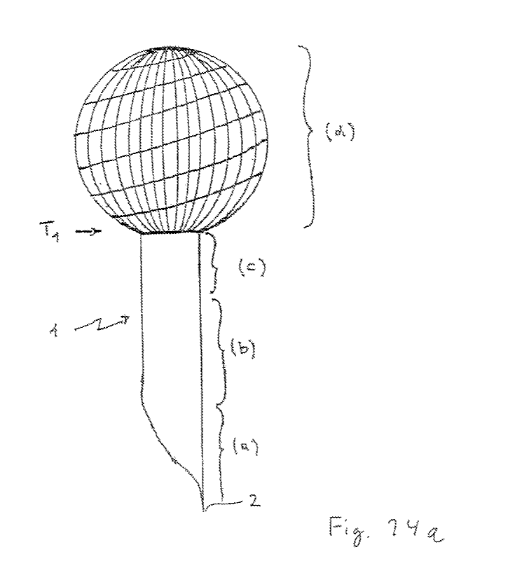

FIG. 13 illustrates another variant of an inventive implant having increased flexibility.

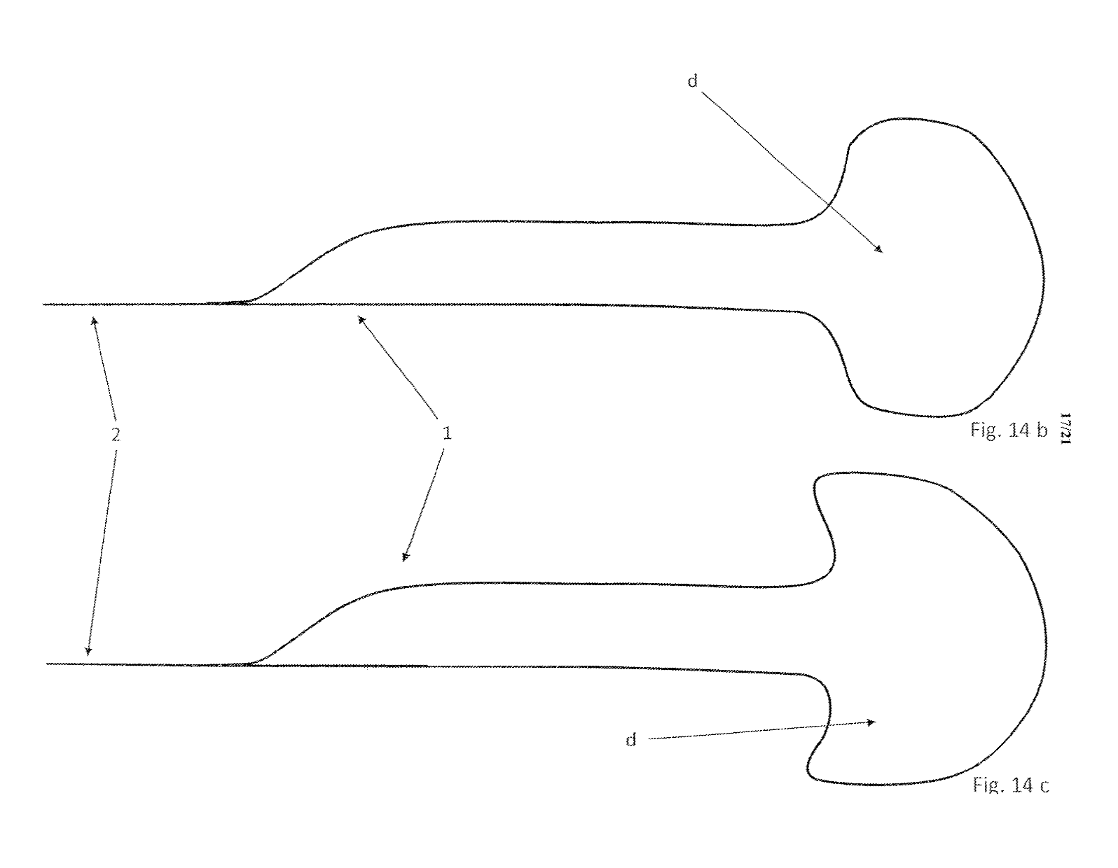

FIG. 14 shows alternative embodiments of an implant according to the invention;

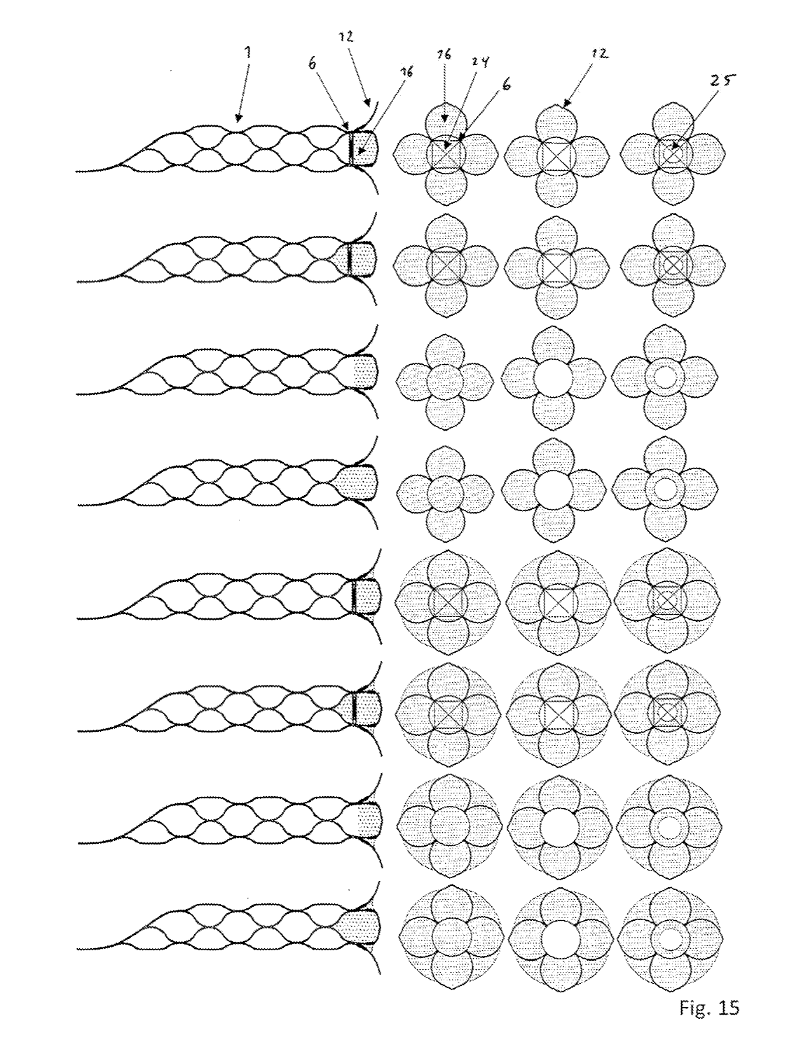

FIG. 15 is a side and a frontal view of several variants of the inventive implant;

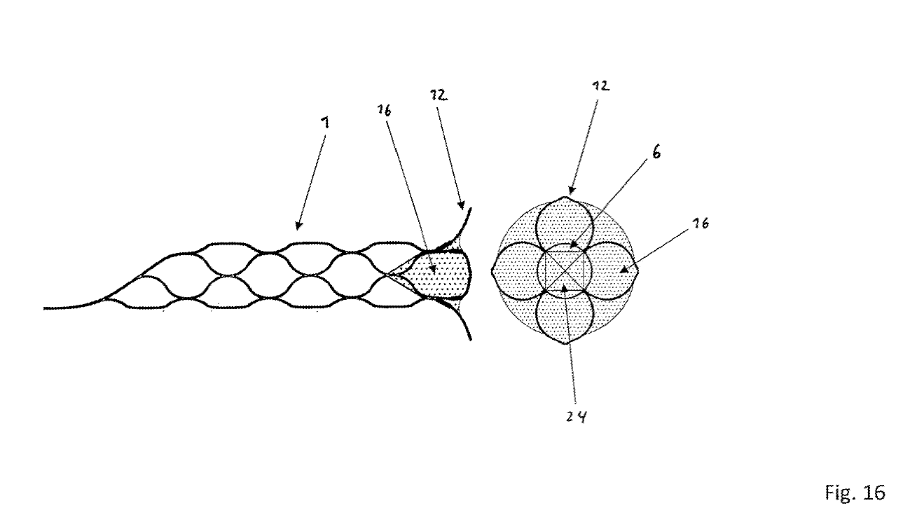

FIG. 16 is a side and a frontal view of another variant of the inventive implant;

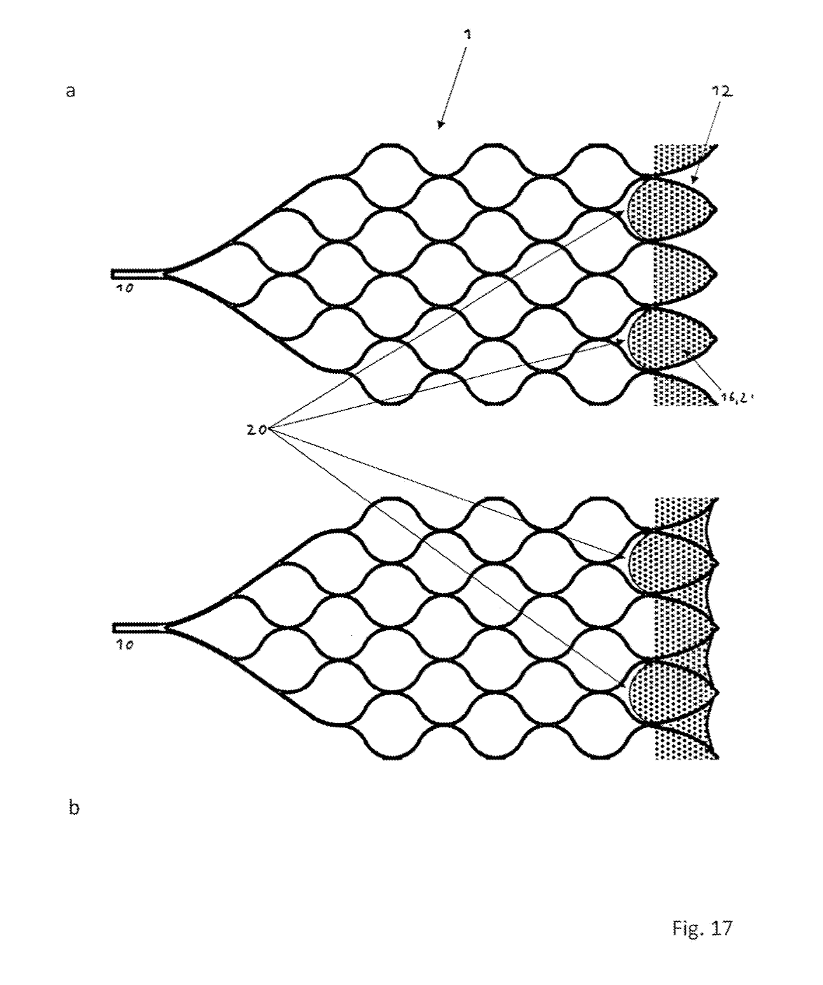

FIG. 17 is a spread planar representation of further variants of the inventive implant, and

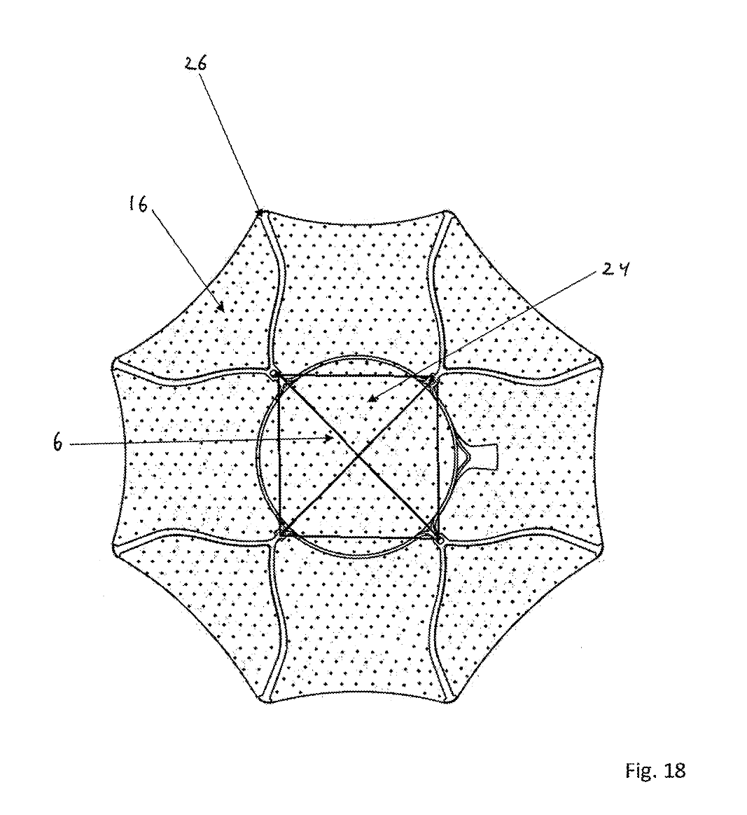

FIG. 18 is a frontal view of another variant of the implant in accordance with the invention.

FIG. 1 illustrates a bifurcation aneurysm with supplying vessel Z, two discharging vessels X and Y as well as aneurysm A situated at the forking location. The long arrows signify the flow of blood into the aneurysm A where it impinges on the aneurysm wall thus exerting outward pressure causing the aneurysm to enlarge (small arrows).

FIG. 2 shows a vessel configuration with an aneurysm A as described in FIG. 1 with an inventive implant 1 being arranged inside the vessel configuration. The implant has a proximal end 2 which is provided with the coupling element and, before detachment, connected to the guidewire (not shown here). By way of its meshes 3 the implant 1 is anchored to the wall of the supplying vessel Z and in the region of the bifurcation has meshes 4 the mesh size of which is greater. A distal region 5 is illustrated in the neck of the aneurysm. Between the distal region 5 and the area where greater meshes 4 are arranged there is a separation zone intended to retain occlusion means introduced into the aneurysm A after the implant has been placed in position.

The enlarged meshes 4 in the area of the bifurcation enable the blood stream inflowing through the supplying vessel Z to be discharged without undue interference via branches X and Y. After occlusion means which are not illustrated here have been introduced into the aneurysm A the flow of blood into aneurysm A is impeded to such an extent that a plug forms inside causing the aneurysm to be blocked off. Alternatively and provided the separation zone is sufficiently impermeable, an occlusion can be achieved without the use of occlusion means.

FIG. 3a is a schematic representation of an inventive implant showing its individual sections.

Implant 1 has a proximal section (a) in which the implant tapers off and terminates in a coupling element, shown here in the form of a wire. This section corresponds to area 2 in FIG. 2.

Distally adjacent to it follows section (b) which serves to secure the implant at the wall of the supplying vessel Z. In this area the size of meshes 3 is relatively narrow so that positive contact with the vessel wall is achieved.

Also in distal direction follows section (c) where meshes 4 are arranged that have a relatively large mesh size. This area is intended to discharge inflowing blood into branches X and Y, see FIGS. 1 and 2 in this respect.

The distal end of the implant 1 is section (d) in which structure 5 in the illustrated case enlarges in a trumpet-like manner. This area will be located within aneurysm A. Section (d) may be an integral part of the implant, i.e. together with sections (a) to (c) be cut out of a tube (of nitinol) or formed into a braiding using wires of this material. However, it is also possible to cut sections (a) to (c) out of a tube, provide a braiding for section (d) and attach said braiding to section (c) by welding.

Separation zone T1 is arranged between sections (c) and (d), said separation zone consisting of one or several separation elements 6. These separation elements may be provided in the form of restrained threads, wires or fibers, for example of polyamide, but may as well consist of parts of a cut structure formed so as to have an inward orientation. Separation zone T1 with separation elements 6 serves to retain the occlusion means introduced into an aneurysm.

Depending on the nature of the aneurysm the separation zone may also be displaced into the section (d) or even be located at the distal end of section (d). Such a separation zone T2 is especially useful if the bifurcation has been deformed in such a way that the efferent vessels X and Y do not directly branch off the supplying vessel Z but instead branch out of the aneurysm. In that case, the separation zone must be located directly above the branches in the dilating section of the implant. Section (d) is limited at the distal end of the implant 1 and extends up to separation zone T2.

FIG. 3b is a representation of the implant proposed by the invention and essentially is similar to what is illustrated in FIG. 3a. It can be seen, moreover, that loops 12 shown here only schematically may form different angles in relation to the longitudinal axis of the implant 1. The longitudinal axis is shown as a broken line. Angle β may be very great (>90°, shown dashed) which is especially helpful with aneurysms of greatly bulging shape wherein the bulge at least partially extends in proximal direction. In extreme cases this angle β may be almost 180°. In this way, the distal section (d) is capable of coming into close contact with the wall of the aneurysm.

In other cases (also shown dashed) it may also be of advantage to arrange for angle β to be negative in the event part of the wall of the aneurysm has an inwardly curved shape. It is to be understood and important that the angles for the individual loops (12)/filaments may differ which offers considerable advantages when treating irregular formed aneurysms.

It can also be seen from FIG. 3b that the proximal end 2 of section (a) where the implant terminates forming coupling wires by means of which the implant is connected to an introducer sheath forms an angle α in relation to the longitudinal axis of the implant. In some circumstances this angle may be formed after implant placement. Not only will the expansion of the implant be improved in this way but implant contact with the wall of the blood vessel is enhanced as well and, furthermore, an undesirable projection into the blood vessel avoided.

FIG. 4 illustrates an inventive implant 1 as it can be put to use as per FIG. 2. The implant 1 is shown to include a guidewire 9 and has been provided with a radiopaque marker coil 7 arranged at its proximal end 2. The coupling element or elements connected to the implant 1 via the guidewire 9 are not shown in the illustration but are arranged in the region of marker coil 7.

The implant shown in the figure is a braiding of individual wires which are preferably made of nitinol and onto which the ultimate form of the implant has been impressed. Nitinol as shape-memory material enables the implant to be inserted into the catheter in compressed form without the shaping of the implant being lost. Having been liberated from the catheter the implant assumes the shape impressed on it so that it can fulfill its purpose as intended.

The implant 1 is divided into four sections (a) to (d), wherein section (a) is the tapering proximal section brought together at the proximal end 2 and terminating in one or several coupling elements. Section (b) has a fixation function and is in contact with the wall of the supplying vessel Z. The section (c) is designed so as to be permeable and provided with meshes 4 through which the flow of blood is allowed to be discharged into efferent vessels X and Y. In comparison with section (b) and also with respect to section (c) the section (d) is of enlarged shape and situated within the aneurysm A. The ends of the individual wires are designed so as to be atraumatic by providing marker coils 8 of a radiopaque material, for example platinum or a platinum alloy. Between sections (c) and (d) a fiber braiding 6 is arranged which may consist of nylon for example and which also forms the separation zone T1. Reference numeral 5 signifies the meshes or filaments in the distal region of the implant 1 that expand outwardly.

FIG. 5 illustrates as a basic principle four design variants of the distal area 5 of implants 1 as proposed by the invention. FIG. 5a shows a distal end of the implant that flares out in a trumpet-like form, i.e. section (d) is designed to form a chalice-shaped enlargement. As illustrated in FIG. 5b the distal end 5 has a disk-like enlargement with distal section (d) being very narrowly limited. FIG. 5c shows a combination of the design elements included in FIGS. 5a and 5b.

FIG. 5d illustrates a distal region where the distal ends of the individual filaments of an implant 1 are rolled up. For better orientation, sections (a), (b), (c) are also referred to in FIG. 5a resp. 5b.

FIG. 6 is the spread planar representation of a preferred embodiment of an inventive implant 1 showing sections (a) to (d). Implant 1 is to be understood as a mesh structure cut out of nitinol tube, wherein the strings 11 shown in the representation as a broken line correspond with the solid-line strings located on the opposite side. The larger honeycombs in the area of section (c) can be easily seen in the representation of FIG. 6a as well as the chalice- or trumpet-shaped enlargement shown in the schematic drawing as per FIG. 6b. Also illustrated there is separation zone T1 with separation elements in the form of an inserted plane composed of nylon threads 6.

FIG. 7a shows an embodiment of the inventive implant as viewed from the distal end. In the distal section (d) loops 12 are arranged that expand radially outwards. Separation zone 6 is formed by a plane consisting of polymer threads or metallic fibers that make sure the occlusion means introduced into the aneurysm are prevented from exiting. Circle 14 symbolizes the transition to the cylindrical part of the implant. Furthermore, the loops are provided with radiopaque marker elements 13.

The separation zone 6, that is, the rectangular framed area in the selected representation, and/or the loops 12 may furthermore be provided with a membrane that effectively blocks the inflowing blood stream into the aneurysm. This membrane may be attached to the polymer threads or metallic fibers as well as the wires of loops 12, and in particular polymer threads or metallic fibers can also be embedded in the membrane. For example, the membrane may consist of polycarbonate urethane and fabricated by means of electrospinning techniques.

FIG. 7b is a side view of the implant 1 of FIG. 7a. In the distal area several loops 12 can be seen that are provided with marker elements 13. Moreover, the entry to the aneurysm is blocked by separation zone 6 which may be formed of polymer threads or metallic fibers interwoven or crossing each other, said threads or fibers preventing occlusion means introduced into the aneurysm from exiting. Loops 12 and/or the separation zone 6 may as well be provided with a membrane cutting off the aneurysm from the flow of blood to a great extent. In this case and circumstances permitting, introducing occlusion means into the aneurysm may be dispensed with. At the proximal end the implant 1 has been provided with a radiopaque marker element 7.

FIG. 7c shows yet another embodiment comprising a membrane 24 arranged in the separation zone 6, with said membrane 24 extending in proximal direction. In particular, the membrane 24 may be of conical or pyramid shape, with the apex of the cone/pyramid being situated proximally. To enable such a membrane to spread out it is considered expedient to reinforce said membrane 24 by means of threads, wires or strings thus keeping membrane 24 in the desired position.

FIG. 8 depicts an inventive implant 1 in which the section (d) has a more disk-like shape, said section consisting for the main part of wire loops 12. Said wire s15 loops are connecting to the cylindrical part of the implant body 1, with this cylindrical portion being composed of the sections (a) to (c). In the transitional area adjacent to the attached loops 12 marker elements 8 are arranged which shall ensure the implant is accurately placed. In the region where the cylindrical body of the implant 1 connects to section (d) in which loops 12 are located there is the section (c) which enables the discharge of the inflowing blood through the laterally efferent vessels. The blood thus enters the efferent vessels (X and Y, FIG. 2) through the spaces between the strings provided with marker elements 8.

Individual variants of the distal section (d) are shown in FIGS. 8b to 8g as a top view representation, wherein individual or several loops 12 can be provided with marker coils 13. The marker coils 13 may embrace the loops either wholly or in part. In the case shown in the figure the loops originate from four connectors 15 that also carry the marker elements 8, with the inner circle 14 constituting the transition to the cylindrical portion of the implant as can be seen from representations 8b to 8g. Any bracings that may exist for a separation zone T1 or T2 are not shown.

The embodiments illustrated in FIGS. 8f and g show loops 12 provided with an extensible membrane 16, said loops simultaneously functioning as a separation zone T2, as depicted in FIG. 3. Moreover, from FIG. 8f it can be seen that each of the loops 12 may be attached to the other areas of the implant via one connecting point only, irrespective of whether loops 12 are provided with a membrane 16 or not.

It is to be understood that separation zones T1 and T2 must partition off the section of aneurysm A that has to be occluded. Depending on the type of aneurysm this separation zone may be situated in the entry region—in the case of vessels branching off proximally to the entry region—or within the aneurysm in the case two vessels branch off out of the aneurysm space itself which in the latter case means only that portion of the aneurysm can be occluded that is free from branching-off vessels. Especially in the event of disk-shaped distal sections (d) of the inventive implants an additional bracing or arrangement of separation elements cut out of the tube may be unnecessary, particularly when a greater number of wire loops has been provided.

Same as the remaining body of the implant the loop-shaped distal sections (d) illustrated in FIG. 8 may, on the one hand, be cut out of a tube of suitable diameter. However, it is also possible to cut sections (a) to (c) of the implant body from a tube in a customary manner and attach to it section (d) consisting of wire filaments, for example by means of a laser welding method.

FIG. 9 shows a special case of an aneurysm A with efferent vessels X and Y branching off out of the aneurysm. In this instance, the implants 1 described by way of FIG. 8 are particularly useful, said implants having loops 12 simultaneously forming the separation zone T2 and, inside the aneurysm itself, being located distally of the branching off vessels. The cylindrical body of the implant 1 with sections (a) and (b) is located within the supplying vessel Z, the section (c) allowing blood to pass through into the branches X and Y is situated in the area of these branches, and section (d) with loops 12 is arranged distally adjacent to said section (c). The loops may be covered by a membrane, with said membrane consisting of an extensible material, for example teflon or a non-woven fabric. Such a non-woven fabric of polycarbonate urethane is known from publication DE 28 06 030 and is characterized by high elasticity conducive to the placement of the implant through a catheter. The membrane may be of slotted, folded or porous design, for instance to save material and facilitate transportation via a catheter.

Such a membrane may also be used as separation element for the separation zone arranged between the sections (c) and (d).

FIG. 10 is a spread out planar representation of several preferred embodiments of an implant 1 according to the invention, wherein the honeycomb structure is composed of honeycombs of substantially equal size, with the exception of the distal loops where the honeycomb surface is larger.

Same as depicted in FIG. 6a the strings 11 shown as a broken line coincide with the solid-line strings on the opposite side. Accordingly, the implant 1 corresponds to a tube having a lattice or honeycomb structure.

Attached to the proximally arranged coupling element 10 follows the proximal section (a) which in turn is followed by the fixation section (b). The distal section (d) starts in the region of eyelets 17 which serve to accommodate and secure wire or nylon elements by means of which a separation zone is arranged within the implant. The distal loops located within the outwardly flared section (d) are distally provided with rounded spouts which are conducive to the positioning of the implant at the placement site via a catheter.

FIG. 10b corresponds in all significant aspects with the representation of FIG. 10a with the exception that a partial slotting is provided in the region of arrows 19, i.e. the location where the tubular structure of the implant 1 is not closed. The slotted configuration extends axially parallel and ends ahead of the distal section (d) at a point where the permeable section (c) is located.

FIG. 10c shows a variant in which a slot 19 is arranged that does not have an axially parallel extension and coils up around the longitudinal axis; however, it also ends ahead of distal section (d).

Slotted arrangements of this nature have proved to be of considerable advantage in terms of flexibility in the area of the fixation zone (b). The radial force of the implant 1 is not significantly impaired in this way but adaptation to the vessel configuration and vessel lumen is improved.

FIG. 10d also shows an inventive implant provided with slots, in this case, however, the slotting does not extend up to the edges of the implant.

Another variant provided with slot 19 is illustrated in FIG. 10e, said slot also coiling up around the longitudinal axis but with honeycomb forms existing side by side. The form of the honeycombs has an effect on the flexibility and can be selected so as to satisfy the relevant needs.

In FIG. 10 all the loops, resp. honeycombs of the distal section (d) are identified by means of reference numeral 12.

FIG. 11 depicts another variant of an inventive implant 1 provided with an individual coupling element 10 and a substantially regular honeycomb structure, wherein additional loops 20 are arranged as separation elements. In the implanted product the additional loops 20 are pointing to the inside and constitute separation zone T1. These loops 20 are also provided with rounded spouts 18 intended to facilitate transportation through a catheter.

FIG. 11b is a schematic representation of the implant shown in FIG. 11a with inwardly pointing loops 20 and the separation zone T1.

FIG. 12 shows another variant of a particularly flexible implant 1 with articulated connectors 21 in the form of a zigzag arrangement of the respective strings provided with a view to improving the adaptation of the implant 1 to curved vessel configurations in the region of the bifurcation.

FIG. 13a illustrates another variant in which as before strings 11 shown as broken line coincide with the solid-line strings on the opposite side. This embodiment is characterized in that the honeycomb structure is interrupted in places, i.e. in some of the honeycombs interruptions, resp. gaps 23 are provided. It is feasible to provide all of the honeycombs with interruptions 23 but in the present Figure some honeycombs only are provided with interruption 23. Moreover, interruptions may be arranged in various sections. In the representation shown here only the honeycombs in section (c) but not those in section (b) are provided with interruptions 23. At the proximal end the implant 1 is attached to the introducer sheath by means of a coupling element 10.

Due to the fact that a retraction into the catheter of an implant 1 that has been provided with interruptions will be difficult or problematic, joining the ends of the implant to form coupling elements at the proximal end may be dispensed with where appropriate. Such an embodiment is illustrated in FIG. 13b. The implant 1 can automatically unfold when being released from the microcatheter.

FIG. 14a shows an embodiment of an implant 1 as proposed by the invention, said embodiment being characterized by a special configuration of the distal s15 section (d) which is shaped in the form of a sphere composed of individual wires or filaments. Starting out from the proximal end 2 of the implant 1 sections (a) to (c) are arranged as described hereinbefore. Between the sections (c) and (d) the separation zone T1 is situated comprising separation elements as they have been described earlier. Instead of an enclosed sphere a distally open basket may also be employed for said section (d). The sphere or the basket shall preferably be of a braided structure.

In FIGS. 14b and c further alternative embodiments are shown for the distal section (d), said embodiments may be referred to as having a spherical or mushroom-like shape which does not necessarily be of regular configuration. In particular, the shape depicted in FIG. 14b deviates from a perfect sphere but is nevertheless capable of fitting closely to the inner wall of the vessel. The embodiment selected in FIG. 14c is particularly suitable for aneurysms of extraordinary form with side walls strongly bulging out and extending partially in proximal direction.

FIG. 15 shows as a side view and viewed from the distal end quite a number of different embodiments of the implant 1 proposed by the invention, wherein membranes 16, 24 being provided in said embodiments. Membranes 16 fill the interior of the wire loops 12, membrane 24 forms (partially) the separation zone 6, wherein the individual membranes 16, 24 may merge in one another or an individual membrane may be provided spanning wire loops 12, separation zone 6, and, as the case may be, further areas. The area provided with a membrane 16, 24 has been shown dotted. As can be seen, areas external to the wire loops 12 may also be spanned by the membrane.

The Figure also shows that there are inventive embodiments wherein a membrane has merely been provided for wire loops 12 whereas the separation zone 6 is formed by threads/wires crossing each other. Additionally, however, the separation zone 6 may also be provided with a membrane 24, and said membrane could then be supported by a thread structure but this is not absolutely necessary. The inner surface of wire loops 12 may be filled by membrane 16 wholly or in part.

Other embodiments are in particular conceivable wherein the membrane 24 forming the separation zone 6 is provided with openings 25 resulting in membrane 24 to be in fact sufficiently impermeable to prevent occlusion means from exiting but still allows a microcatheter to be inserted into the aneurysm through opening 25 with a view to introducing occlusion means. In the event crossing threads/wires are still arranged in the area of separation zone 6 an adequate space must be left free to enable a catheter to pass through.

A similar representation of an alternative embodiment of the inventive implant 1 is shown in FIG. 16, wherein membrane 24 forming the separation zone 6 has a pyramid shape extending in proximal direction. In this manner, the blood stream can be diverted sideways, that is, away from the centrally located aneurysm. Membrane 24 is secured by a thread structure with a fixation proximal to separation zone 6 which enables said pyramid shape to be brought about. As soon as the implant 1 is retracted into a catheter membrane 24 is also pulled further proximally and collapses as a result of which the cross sectional load reduces. Despite a sufficiently impermeable separation zone 6 in expanded state an implant is produced in this way that can be maneuvered through a suitable catheter without difficulty.

FIG. 17 shows an embodiment wherein, similar to what is illustrated in FIG. 11, the separation zone 6 is formed by wire loops 20 facing towards the interior. The Figure is a side view of a representation showing an unfolded implant structure. The representation shows two inwardly projecting wire loops 20, it is to be understood, however, that more wire loops 20 may be provided. Same as wire loops 12 which are distally projecting outwardly the wire loops 20 are provided with a membrane 16, 24 that further increases in the area of the separation zone 6 the impermeability of said zone 6. Moreover, in the variant shown in FIG. 17b also the interspaces between wire loops 12 are provided with a membrane 16.

FIG. 18 is a frontal view of another inventive embodiment. The representation is similar to that shown in FIG. 15 but instead of loops braces 26 radially pointing outwardly are provided in this case and form section (d). The braces 26 converge concentrically, with two braces 26 each forming a unit and with said two braces sharing a common origin at the distal end of section (c). Braces 26 furthermore serve to spread out a membrane 16, 24 (shown dotted) extending over both the inner area of the separation zone and the interspaces between the braces 26. A thread structure 6 can be arranged in the separation zone to additionally improve the stability but this is not strictly necessary. However, such a thread structure 6 facilitates penetration of individual segments of the inner membrane 24, whereas at the same time other areas of membrane 24 remain undamaged which makes it possible to introduce occlusion means into the aneurysm.

1. Implant having a mesh structure (3, 4) to be used for the occlusion of aneurysms in the area of vessel branches, particularly bifurcation aneurysms (A), which—from proximal to distal—is provided with sections (b) to (d):
(b) a fixation section by means of which the implant can be supported on a vessel wall,
(c) a permeable section for the vessel branching area, and
(d) a distal section where in comparison with section (b) the implant is expanded radially and which is meant to be placed into the aneurysm (A),
wherein in the area of sections (c) or (d) a separation zone (T1, T2) is arranged that closes off at least partially the neck of the aneurysm, the distal section (d) being provided with a plurality of filaments and/or loops (12) connecting to section (c) and said filaments/loops (12) forming an angle of between −45° and +175° in relation to the longitudinal axis of the implant (1), wherein a positive angle is indicative of filaments/loops pointing radially outward and a negative angle of filaments/loops (12) pointing radially inward.

2. Implant according to claim 1, characterized in that the filaments/loops (12) form an angle of between +45° and +90° in relation to the longitudinal axis of the implant (1).

3. Implant according to claim 1, characterized in that the filaments/loops (12) in the distal section (d) are provided with eyelets (17).

4. Implant according to claim 1, characterized in that the filaments/loops (12) in the distal section (d) are provided with rounded spouts (18) at the distal end.

5. Implant according to claim 1, characterized in that the loops (12) are provided inside with a membrane (16) or that a membrane (16) is spanned between the filaments.

6. Implant according to claim 1, characterized in that the filaments/loops (12) are attached to section (c) by means of individual, in particular one or two connecting points.

7. Implant having a mesh structure (3, 4) to be used for the occlusion of aneurysms in the area of vessel branches, particularly bifurcation aneurysms (A), which—from proximal to distal—is provided with sections (b) to (d):
(b) a fixation section by means of which the implant can be supported on a vessel wall,
(c) a permeable section for the area of vessel branching, and
(d) a distal section where in comparison with section (b) the implant is expanded and which is meant to be placed into the aneurysm (A),
wherein in the area of sections (c) or (d) a separation zone (T1, T2) is arranged that at least partially closes off the neck of the aneurysm, with said distal section (d) taking on an enlarged shape similar to a sphere, mushroom, anchor or ellipsoid.

8. Implant according to claim 1, characterized in that the implant (1) proximal to section (b) is provided with a section (a), wherein said section (a) is a tapering proximal section in which the mesh structure (3, 4) is brought together in the form of one or several coupling elements (10), preferably coupling wires.

9. Implant according to claim 8, characterized in that the coupling elements (10) are brought together eccentrically on the periphery of the implant (1) in its expanded form.

10. Implant according to claim 9, characterized in that the coupling elements (10), preferably coupling wires, form an angle ranging between 0° and +60°, preferably +10 to +30°, in relation to the longitudinal axis of the implant (1), with a positive angle being indicative of coupling elements (10) pointing outward.

11. Implant according to claim 1, characterized in that the separation zone (T1, T2) is provided with separation elements consisting of filaments (6) arranged orthogonally to the longitudinal axis of the implant (1), preferably extending substantially in a plane.

12. Implant according to a claim 1, characterized in that the separation zone (T1, T2) is provided with a membrane (24) spanning orthogonally to the longitudinal axis of the implant (1).

13. Implant according to claim 12, characterized in that the membrane (24) is secured to filaments (6) arranged in the separation zone (T1, T2).

14. Implant according to claim 12, characterized in that the membrane (24) extends in proximal direction and, preferably, has a conical or pyramid form.

15. Implant according to claim 12, characterized in that the membrane (24) has one or several openings (25) or that in the membrane (24) one or several openings can be produced by a piercing method.
